(19) United States
(12) Patent Application Publication
(10) Pub. No.: US 2013/0225790 A1
(43) Pub. Date: Aug. 29, 2013

(54) MOMORDICA CHARANTIA POLYPEPTIDE, PREPARATION METHOD THEREOF AND USES OF THE SAME

(71) Applicant: Yuandong Zou, Hubei (CN)

(72) Inventor: Yuandong Zou, Hubei (CN)

(21) Appl. No.: 13/881,353

(22) PCT Filed: Oct. 21, 2011
(86) PCT No.: PCT/CN11/01758

§ 371 Date: Apr. 24, 2013

(30) Foreign Application Priority Data
Oct. 25, 2010 (CN) 201010526066.6

(51) Int. Cl. C07K 4/10; C12P 21/00

(52) U.S. Cl. CPC C07K 4/10; C12P 21/00
USPC 530/300; 435/272

(57) ABSTRACT

A Momordica charantia polypeptide, preparation method thereof and uses of the same are provided. This polypeptide is prepared according to the following steps: the bitter gourd powder is enzymolysed with papain to give a polypeptide with a molecular weight of 130˜1000 Da. The resultant polypeptide consists of 2-9 amino acids, is easily absorbed by human body, and possesses the biological function of balancing blood glucose. The Momordica charantia polypeptide provided in the present application may be used for preparation of a medicine, food or food additives for the prophylaxis and/or treatment of diabetes.

### FIELD OF THE INVENTION

The present invention relates to a Momordica charantia polypeptide, preparation method thereof and uses of the same.

### DESCRIPTION OF BACKGROUND

Momordica charantia L. is a member of the Cucurbitaceae family All parts of the plant, including the fruit, taste bitter. The plant is characterized by toothed leaf margin, and oblong fruits, with young fruit emerald green, turning to orange-yellow when ripe. This plant is grown in subtropical area and widely planted throughout the world as a vegetable and medicine. The fruit thereof is bitter in flavor, cold in nature, and possesses pharmaceutical effects such as clearing away heat and diminishing inflammation, improving eyesight and detoxicating toxic material (removing toxicity), which is used to treat a trauma by topical external application, and to deworm, regulate menstruation, resist viruses, and treat measles and hepatitis by internal application. It is confirmed to be effective in treating diabetes and have the anti-viral, anti-cancer, anti-bacterial and insecticidal effects by pharmacological experiments.

At present, researchers both from home and abroad are interested in treating diabetes with bitter gourd. Many products, such as “momordicin”, “total saponins from bitter gourd”, “bitter gourd tea”, “bitter gourd powder” and the like are developed sequentially. However, the effective ingredients in these products are relatively large in molecular weight, which are not easily absorbed and utilized by human body. Based on the theory of traditional Chinese medicine, it is unsuitable for people of cold body type to eat or eat more or eat frequently the products, such as “bitter gourd powder”, “bitter gourd tea”, and “momordicin”.

### DISCLOSURE OF THE INVENTION

The object of the present invention is to provide a Momordica charantia polypeptide and preparation method thereof.

The Momordica charantia polypeptide provided by the present invention is obtained according to the method comprising the step of: an enzymolysis reaction is conducted on the bitter gourd powder with papain to give said Momordica charantia polypeptide.

Said bitter gourd powder in the present invention is obtained by lyophilizing the fresh bitter gourd prior to pulverizing.

Said lyophilization may be conducted according to conventional methods, for example, drying for 12 h in a lyophilizer (pre-freezing stage): the temperature inside the lyophilizer is firstly decreased to −30° C. to −40° C. for 2˜3 h; sublimation stage: a vacuum is drawn on the whole system when the temperature of the chiller drops below −30° C., with the pressure set between 15˜20 Pa, and the temperature raised to 30˜35° C. for 10˜15 h; secondary sublimation stage: the temperature is raised to 45˜50° C. for 3˜5 h to give a lyophilized bitter gourd.

In the present invention, said enzymolysis reaction is performed under the temperature of 38-42° C. for a period of 7-8 hours.

In the reaction system of the enzymolysis reaction, the ratio, parts by weight, of the bitter gourd powder and water is 1:8-10; 150000-180000 U of papain is needed for enzymolysis of 1 g bitter gourd powder. The enzyme activity unit, U, refers to the amount of enzyme needed for converting 1 μmol of substrate per minute or converting 1 μmol of corresponding groups per minute under specific conditions (25° C., pH7.0).

When the enzymolysis reaction finishes, the enzymolysis solution is further subjected to an enzyme inactivation treatment under the following condition: the temperature is 100° C., with a period of 30 min. Then, cryopreservation and sedimentation treatment is conducted, under 2-4° C. for a period of 48-72 h, to give a purified Momordica charantia polypeptide concentrated solution.

For convenient storage and transportation and applicable in more dosage forms, the above-described Momordica charantia polypeptide concentrated solution may further be subjected to lyophilization or spray drying to yield Momordica charantia polypeptide powders.

The Momordica charantia polypeptide obtained according to the above-described method has a relative molecular weight of 130-1000 Da, which is comprised of 2-9 amino acids, and is excellent in nutrition and hypoglycemic physiological activity. This Momordica charantia polypeptide may be present in the form of a concentrated solution, a lyophilized dry powder, or a spray dried dry powder.

Another object of the present invention is to provide uses of said Momordica charantia polypeptide.

Use of the Momordica charantia polypeptide provided by the present invention is a use of the same in manufacturing a product for the prophylaxis and/or treatment of diabetes.

Still another object of the present invention is to provide a product for the prophylaxis and/or treatment of diabetes.

The present invention provides products for the prophylaxis and/or treatment of diabetes with the Momordica charantia polypeptide provided by the present invention as the active ingredient thereof. The products may include medicines, healthcare products, or food etc.

Into the medicine or healthcare product for prophylaxis and/or treatment of diabetes, prepared with the Momordica charantia polypeptide as the active ingredient, one or more pharmaceutically acceptable carriers may further be added if necessary. The carrier includes conventional diluents, excipients, fillers, adhesives, humectants, disintegrants, absorption enhancers, surfactants, adsorption carriers, lubricants and the like in the pharmaceutical field.

The medicine for the prophylaxis and/or treatment of diabetes may be prepared into multiple forms, such as oral liquid, tablet, granules, capsule (including soft and hard capsules), spray and coated pill and the like. The medicines in the above various dosage forms may be prepared according to conventional methods in the pharmaceutical field.

### BRIEF DESCRIPTION OF THE DRAWINGS

FIG. 1 is a process flow diagram for preparing the Momordica charantia polypeptide of the present invention.

FIG. 2 is a gel chromatogram of the Momordica charantia polypeptide in Example 4.

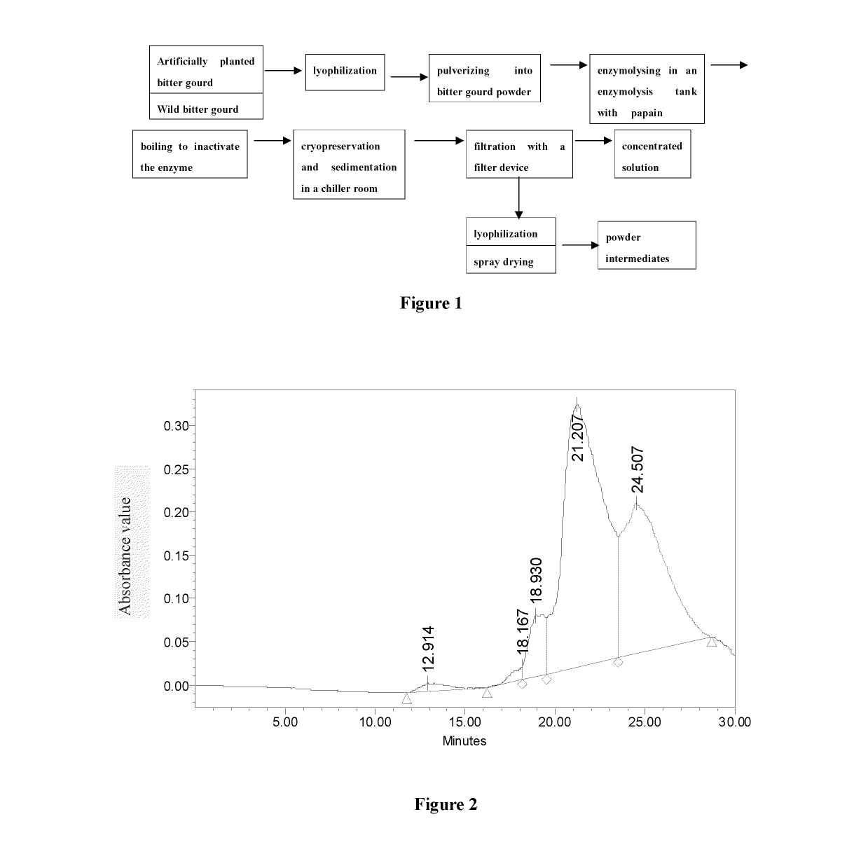

### BEST MODE FOR CARRYING OUT THE INVENTION

Hereafter, the method of the present invention will be explained with specific Examples; however, the present invention is not limited to thereto.

Each of the experimental methods in the following Examples is a conventional method, unless otherwise indicated. All of the reagents and biological materials are commercially available, unless otherwise indicated.

The process for lyophilizing the fresh bitter gourd in the following Example comprises: drying for 12 h in a lyophilizer (pre-freezing stage): the temperature inside the lyophilizer is firstly decreased to −30° C. to −40° C. for 2˜3 h; sublimation stage: a vacuum is drawn on the whole system when the temperature of the chiller drops below −30° C., with the pressure set between 15˜20 Pa, and the temperature raised to 30˜35° C. for 10˜15 h; secondary sublimation stage: the temperature is raised to 45˜50° C. for 3˜5 h to give a lyophilized bitter gourd.

### EXAMPLE 1

### Preparation of Momordica charantia Polypeptide

The fresh bitter gourd (artificially planted or wild polyspermous bitter gourd) was lyophilized in a lyophilization device, and then, pulverized into powders to give the lyophilized bitter gourd powders. Next, the bitter gourd powders were put into an tank for enzymolysis, into which water, nine times the mass of the bitter gourd powders, was added, together with the papain in an amount of 165000 U of papain per gram of bitter gourd powders. The bitter gourd powders were enzymolysed for 8 h under the temperature of 40° C. After enzymolysis, the enzymolysis solution was subject to an enzyme inactivation treatment under the temperature of 100° C. for a period of 30 min followd by a cryopreservation and sedimentation treatment at the temperature of 2-4° C. for 60 h, and filtration was performed with a filtration device three times when the impurities therein were substantially precipitated, producing the Momordica charantia polypeptide concentrated solution. This Momordica charantia polypeptide concentrated solution was subjected to lyophilization or spray drying treatment, giving the Momordica charantia polypeptide powders.

### EXAMPLE 2

### Preparation of Momordica charantia Polypeptide

The fresh bitter gourd (artificially planted or wild polyspermous bitter gourd) was lyophilized in a lyophilization device, and then, pulverized into powders to give the lyophilized bitter gourd powders. Next, the bitter gourd powders were put into an tank enzymolysis, into which water, eight times the mass of the bitter gourd powders, was added, together with the papain in an amount of 150000 U of papain per gram of bitter gourd powders. The bitter gourd powders were enzymolysed for 7 h under the temperature of 38° C. After enzymolysis, the enzymolysis solution was subject to an enzyme inactivation treatment under the temperature of 100° C. for a period of 30 min followed by a cryopreservation and sedimentation treatment at the temperature of 2-4° C. for 48 h, and filtration was performed with a filtration device three times when the impurities therein were substantially precipitated, producing the Momordica charantia polypeptide concentrated solution. This Momordica charantia polypeptide concentrated solution was subjected to lyophilization or spray drying treatment, giving the Momordica charantia polypeptide powders.

### EXAMPLE 3

### Preparation of Momordica charantia Polypeptide

The fresh bitter gourd (artificially planted or wild polyspermous bitter gourd) was lyophilized in a lyophilization device, and then, pulverized into powders to give the lyophilized bitter gourd powders. Next, the bitter gourd powders were put into an enzymolysis tank, into which water, ten times the mass of the bitter gourd powders, was added, together with the papain in an amount of 180000 U of papain per gram of bitter gourd powders. The bitter gourd powders were enzymolysed for 8 h under the temperature of 42° C. After enzymolysis, the enzymolysis solution was subject to an enzyme inactivation treatment under the temperature of 100° C. for a period of 30 min followed by a cryopreservation and sedimentation treatment at the temperature of 2-4° C. for 72 h, and filtration was performed with a filtration device three times when the impurities therein were substantially precipitated, producing the Momordica charantia polypeptide concentrated solution. This Momordica charantia polypeptide concentrated solution was subjected to lyophilization or spray drying treatment, giving the Momordica charantia polypeptide powders.

### EXAMPLE 4

### Determination of the Molecular Weight Distribution of Momordica charantia Polypeptide

The molecular weight distribution of Momordica charantia polypeptide was determined using high performance gel filtration chromatography, and detected by the Analysis and Test Center of Jiangnan University.

The specific method used was as follows:

1. Summary of the Method

Determination was made by high performance gel filtration chromatography. That is, using porous fillers as a solid phase, isolation was made according to the size differences of the molecular volume of the components in the sample. Detection was conducted under the UV absorption wavelength of the peptide bond of 220 nm, and a data processing software special for determining the molecular weight distribution with gel chromatography (i.e., the GPC software) was used to process the chromatograms and data thereof, and then, the relative molecular mass of the Momordica charantia polypeptide and distribution range thereof were calculated and obtained.

2. Machines

a) High performance liquid chromatography equipped with a UV detector and a chromatography workstation or integrator containing the GPC data processing software;

b) Mobile phase vacuum filtration degasser;

c) Ultrasonic oscillator;

d) Analytical balance, 0.0001 g sensitivity.

3. Reagents

a) Acetonitrile, chromatographic pure; b) trifluoroacetic acid, analytical pure; c) water, ultrapure grade or double distilled water.

d) Standards used for molecular weight calibration curve:

1) cytochrome C (cyyochrome, MW12500); 2) aprotinin (MW6500)

3) bacillus enzyme (bacitracin, MW1450); 4) ethyl amino acid-ethyl amino acid-tyrosine-arginine (NW451)

5) ethyl amino acid-ethyl amino acid-ethyl amino acid (MW189)

4. Chromatographic Conditions and System Suitability Test

Chromatographic column: TSKge1G2000 SWXL 300 mm×7.8 mm, or other gel column of the same type, which has similar performance, and which is suitable for determining proteins and peptides.

Mobile phase:acetonitrile:water:trifluoroacetic acid, 10:90:0.1 (by volume)

Detected wavelength: UV220 nm

Flow rate: 0.5 mL/min

Column temperature: 30° C.

Sample volume:10 μL

It was specified that, under the above chromatographic conditions, the column efficiency of the gel chromatography, i.e., the theoretical plate number (N) should be no less than 10000 when calculated based on the tripeptide standard (ethyl amino acid-ethyl amino acid-ethyl amino acid) peak, and the partition coefficient (Kd) of Momordica charantia polypeptide should be between 0-1, so that the chromatography system satisfies the testing requirements.

5. Preparation of the Molecular Weight Calibration Curve

The mobile phases were used separately to formulate a 0.1 wt % standard solution of the above peptides with different molecular weights, a polytetrafluorethylene or nylon filtration membrane with a pore diameter of 0.2 μm˜0.5 μm was used and then the sample is injected separately to give the chromatograms of a series of standards.

The logarithm of the molecular weight (1 gMV) was used to make a plot or a linear regression on the retention time to give the molecular calibration curve and the equation thereof.

6. Preparation of the Sample

Samples, weighed about 20.0 mg, were put into a 10 ml volumetric flask, and the width was brought to the scale with the mobile phase. Samples were sufficiently dissolved and mixed via ultrasonic vibration for 10 min, and injected into the machine after filtered through a polytetrachloroethylene or nylon filtration membrane with a pore diameter of 0.2 μm˜0.5 μm.

7. Calculation of the Molecular Weight Distribution

The sample solution prepared in 6 was analyzed under the above chromatographic conditions, see FIG. 2 for the chromatogram. Then, calculation was made by substituting the chromatography data of the samples into the calibration curve with the GPC data processing software to give the molecular weight of the samples and distribution range thereof.

The relative percentage of the peptides with different molecular weight ranges may be obtained with the peak area normalization method.

TABLE 1
Determination result of the molecular weight distribution
of the Momordica charantia polypeptide

| | Percentage of | | |
|---|---|---|---|
| Range of the | the peak area | Number average | Weight average |
| molecular weight | (%, λ 220 nm) | molecular weight | molecular weight |
| >5000 | 2.05 | 6590 | 7056 |
| 5000~2000 | 6.47 | 3745 | 3791 |
| 2000~1000 | 15.39 | 1308 | 1364 |
| 1000~500 | 30.10 | 676 | 708 |
| 500~130 | 36.34 | 282 | 313 |
| <130 | 9.65 | / | / |

After detection by the Analysis and Test Center of Jiangnan University, 23.91% of the Momordica charantia polypeptides prepared in Examples 1˜3 have a relative molecular mass >1000 Da, and 66.44% have a relative molecular mass of 130-1000 Da, see Table 1 for the detailed results. The results indicate that the relative molecular mass of the Momordica charantia polypeptides provided by the present invention is mainly in the range between 130-1000 Da.

### EXAMPLE 5

### Pharmacodynamic Trial of the Momordica charantia Polypeptide

Animal testing was conducted on a Type II diabetes rat model with the Momordica charantia polypeptide prepared in Example 1. Therapeutic effect of the Momordica charantia polypeptide of the present invention on the rats tested was inspected by using controlled trials.

The specific experimental process was as follows:

Rats were fed with basal feed for a week, then, randomly divided into the normal feed group (normal control group, 10 rats) and the high fat feed group (60 rats), and continuously fed for another 8 weeks, respectively. After that, rats of the high fat feed group received a single intravenous injection of STZ (streptozotocin), 25 mg/kg, via tail vein, and rats of the normal control group received an injection of sodium citrate-citric acid buffer of equal amount. The rats with 12 mmol/L≦blood glucose<30 mmol/L after 2 d were regarded as being successful type II diabetes rat models. 40 2-DM rats, succeeded in modeling, were randomly divided into four groups: model control group (given water by gavage), and high, medium, and low dosage groups in Example 1 (the administration dosage of the high dosage group is 100 mg/kg, of the medium dosage group is 50 mg/kg, and of the low dosage group is 25 mg/kg, gavage administration). Gavage administration was continued for 8 days, then, animals were observed for symptoms and detected for various indexes. Rats were determined for fasting blood glucose and other indexes after the experiment finished.

SPSS 16.0 statistical software was used for data processing; the experimental data was represented by mean±standard deviation(±s); t-test was employed, with a significance level α=0.05.

TABLE 2
Effects of Momordica charantia polypeptide on blood glucose
of the type II diabetes rat (χ ± SD)

| | | Before | Post- | Percentage |
|---|---|---|---|---|
| | | experiment | experiment | of decline |
| Group | n | (mmol/L) | (mmol/L) | in mean (%) |
| Blank control group | 10 | 5.69 ± 0.96 | 5.36 ± 0.27 | |
| Model control group | 10 | 12.69 ± 0.46 | 11.89 ± 0.86 | |
| Example 1-low | 10 | 12.27 ± 0.66 | 9.77 ± 1.46* | 20% |
| Example 1-medium | 10 | 12.83 ± 1.04 | 8.01 ± 1.22* | 37.6% |
| Example 1-high | 10 | 11.88 ± 1.09 | 7.09 ± 1.13* | 40.3% |

Compared with the model control group,
*P < 0.05, indicating a significant difference.

TABLE 3
Effects of Momordica charantia polypeptide on insulin
and hepatic glycogen of the type II diabetes rat

| | | Serum insulin | hepatic glycogen |
|---|---|---|---|
| Group | n | (uIU/L) | (mg/g) |
| Blank control group | 10 | 60.69 ± 10.96 | 25.36 ± 4.27 |
| Model control group | 10 | 97.2 ± 16.44 | 13.23 ± 3.86 |
| Example 1-low | 10 | 72.2 ± 23.54* | 18.77 ± 5.46* |
| Example 1-medium | 10 | 68.8 ± 21.04* | 21.01 ± 6.22* |
| Example 1-high | 10 | 60.8 ± 19.09* | 24.09 ± 4.13* |

Compared with the model control group,
*P < 0.05, indicating a significant difference.

TABLE 4
Effects of Momordica charantia polypeptide on
blood SOD and MDA of the type II diabetes rat

| | | SOD | MDA |
|---|---|---|---|
| Group | n | (u/mL) | (nmol/ml) |
| Blank control group | 10 | 159.73 ± 17.48 | 8.36 ± 2.27 |
| Model control group | 10 | 100.24 ± 16.44 | 26.23 ± 1.86 |
| Example 1-low | 10 | 119.27 ± 33.54 | 20.07 ± 2.46 |
| Example 1-medium | 10 | 132.83 ± 21.04 | 15.71 ± 1.22 |
| Example 1-high | 10 | 152.88 ± 21.09 * | 12.93 ± 1.13 * |

Compared with the model control group,
* P < 0.05, indicating a significant difference.

In summary, the present experimental results indicate that: the Momordica charantia polypeptide has significant hypoglycemic effects, can improve the insulin sensitivity of organisms with diabetes, increase glucose utilization of liver, and improve glucose metabolism of organisms. Further, the Momordica charantia polypeptide can notably reduce the content of MDA in serum, and remarkably increase the SOD activity, thereby possessing certain medical and pharmaceutical values.

### INDUSTRIAL APPLICATION

The present invention used the bitter gourd powder as a substrate and provided the Momordica charantia polypeptide by enzymolysis of the bitter gourd powder with papain. This product may serve as a pharmaceutical raw material and food additive for preparing a product for the prophylaxis and/or treatment of diabetes such as medicine, healthcare product, food and the like.

1. A method for preparing a Momordica charantia polypeptide, comprising:
conducting an enzymolysis reaction on bitter gourd powder with papain to obtain the Momordica charantia polypeptide.

2. The method according to claim 1, wherein the bitter gourd powder is obtained by lyophilizing a fresh bitter gourd prior to pulverizing.

3. The method according to claim 1, wherein the enzymolysis reaction takes place at a temperature of 38-42° C. for 7-8 h.

4. The method according to claim 1, wherein in the reaction system of the enzymolysis reaction, the a ratio, parts by weight, of the bitter gourd powder and water is 1:8-10; and 150000-180000 U of papain is needed for enzymolysing 1 g of the bitter gourd powder.

5. The method according to claim 1, wherein the method further comprises:
after the enzymolysis reaction, subjecting a solution for enzymolysis to a cryopreservation and sedimentation treatment under the temperature of 2-4° C. for 48-72 h;
filtering the solution after the cryopreservation and sedimentation treatment; and
collecting a filtrate.

6. The method according to claim 5, wherein the method further comprises subjecting the filtrate to an enzyme inactivation treatment, wherein a condition for the enzyme inactivation is: temperature of 100° C., and time of 30 min.

7. The Momordica charantia polypeptide prepared by the method according to claim 1.

8. (canceled)

9. (canceled)

10. A product for the prophylaxis and/or treatment of diabetes, having the Momordica charantia polypeptide of claim 7 as an active ingredient thereof.
